(19) United States
(12) Patent Application Publication
(10) Pub. No.: US 2001/0004682 A1
(43) Pub. Date: Jun. 21, 2001

(54) Needleless injector drug capsule

(75) Inventor: Terence Edward Weston, Eye (GB)

Correspondence Address: DARBY & DARBY P.C., 805 Third Avenue, New York, NY, 10022, US

(73) Assignee: Weston Medical Ltd.

(21) Appl. No.: 09/731,398

(22) Filed: Dec. 1, 2000

Related U.S. Application Data
(63) Continuation-in-part of application No. 09/091,320, filed on Aug. 5, 1998.
Application No. 09/091,320 is a 371 of international application No. PCT/GB96/03017, filed on Dec. 9, 1996.

(30) Foreign Application Priority Data
May 19, 2000 (GB) PCT/GB00/01922
Dec. 16, 1995 (GB) 9525757.2
May 19, 2000 (GB) 9911663.4
Oct. 15, 1999 (GB) 9924533.4

(51) Int. Cl. A61M 5/30

(52) U.S. Cl. 604/72

(57) ABSTRACT

A transparent needleless injector drug capsule suitable for prefilling with a liquid drug comprises a first inner layer of drug compatible transparent plastics defining a chamber for receiving a liquid drug and a second outer layer of transparent plastics forming a supporting sleeve around the first layer of plastics. Each of the first and second layers of transparent plastics is resistant to discoloration when irradiated by high energy radiation. Thus, a low cost, transparent, needleless injector drug capsule is provided which is capable of being sterilized by high energy irradiation. The capsule may define a body having a main chamber for receiving a liquid drug and for retaining a free piston in a sealing fit for subsequent use in the discharge of a drug, the body also having an extension chamber having an opening for receiving the free piston in a loose fit. The initial loose fitting of the piston allows penetration by a sterilization fluid, so that the drug capsule is capable of being assembled in clean conditions and subsequently sterilized by any known method.

### BACKGROUND TO THE INVENTION

Needleless injectors are used as an alternative to needle-type hypodermic injectors for injecting liquid drugs through the epidermis and into the underlying tissues. The normal form of construction is a syringe having a small discharge orifice which is placed on the skin, and through which the drug is discharged at sufficiently high pressure to puncture the skin. The force required to pressurise the drug may be derived from a compressed coil spring, compressed gas, explosive charge or other form of stored energy.

It is an advantage if the pressure induced in the drug during the injection cycle comprises a first phase in which the pressure rises rapidly to a peak, followed by a second phase at lower pressure. The first phase facilitates the penetration of the skin by the drug and the second phase dispenses the drug through the hole thus formed. Typically, the pressure reached in the first phase is in the order of 300-400 bars, with a rise time of about 50 μs, whilst the second phase is completed at a pressure of about 100 bars. Using an injection orifice of 0.5 mm the injection cycle for 500 μl of drug is between 30 and 50 ms for water or liquids having similar characteristics to water.

The capsule from which the drug is discharged is often in the form of a cylinder containing a free piston (ie no connecting rod), with the discharge orifice located in an end wall. The orifice may be formed integrally with the cylinder or there may be a separate nozzle in sealing hydraulic contact with the end of the cylinder. The other end of the cylinder may be open to receive a driving push rod which acts on the piston to cause the discharge of drug. The complete injector may be presented as a single use, pre-filled and disposable device; or as a multiple use actuator with replaceable drug capsules; or as a multi-dose actuator which dispenses successive doses from a bulk supply.

Needleless injectors place heavy demands on the capsule construction because of the extremely high stresses induced during injection. The materials used must be strong, highly transparent so that the drug may be checked visually or by laser inspection instruments for contamination and entrapped gas, and be chemically compatible with the drug to be stored. The ideal material for the capsule is so-called type 1 borosilicate glass, which is very commonly used for needle-type syringes that are pre-filled with drug. Whilst glass capsules made to the appropriate specifications have excellent performance, they require rigorous proof testing to eliminate glass containing common flaws such as entrained bubbles and foreign matter, cracks and scratches, and they also require time consuming cleaning and sterilisation before they can be filled. Typically, glass capsules are first washed to remove particles and then dry heat sterilised and depyrogenated by heating to around 180° C. for at least 6 hours. Glass capsules are therefore expensive to make.

Recently, some manufacturers have turned to using plastics instead of glass. Plastics technology allows more precise control of the capsules' dimensions at high production rates. However, plastics materials introduce a number of problems which are not found in glass, one of which is the problem of drug compatibility. In general, plastics are not suitable for long term drug contact. Plastics are gas permeable, absorb water and contain material which can adversely react with the drug. Furthermore, plastics injection moulding technology typically requires the use of release agents which are not drug compatible requiring time consuming and expensive cleaning and sterilisation of the plastics drug capsule. Many drugs are sensitive to oxygen and so gas permeability must be minimised. The absorption of water would change the concentration of a liquid drug. Finally, many transparent plastics are quite brittle and therefore not capable of withstanding the extremely high stress induced during injection. Furthermore, very little is known about the effects of high strain rates as experienced with needleless injectors. Indeed, datasheets provided by manufacturers only disclose mechanical properties based on standard tensile strength and impact texts carried out at relatively slow strain rates.

If it is necessary to ensure a very low burden of pyrogens on containers for storing parenteral drugs, pyrogen free moulding technology combined with gas sterilisation, typically using ethylene oxide, may be used in place of dry heat sterilisation. The use of gas sterilisation precludes the preassembly of the free piston in the drug capsule to ensure all surfaces can be reached. Furthermore, some plastics are sensitive to the sterilisation gas. The use of ethylene oxide is banned in some countries due to public health concerns.

In view of the above, it is not surprising that the use of plastics drug capsules for needleless injectors is restricted to applications where the drug capsule is filled immediately before use, generally with the aid of a transfer device which enables the drug to be drawn from a vial and transferred to the injector capsule. This is a tedious operation and it is easy to make mistakes by filling the wrong dose, and trapping air in the drug. Furthermore, aseptic and pyrogen-free transfer of drug is very difficult to achieve in the everyday environment.

There is a growing need for a single use, pre-filled needleless injector, which is simple to use and safely disposable, and thus have the advantage of factory control of the drug filling process.

### SUMMARY OF THE INVENTION

According to a first aspect of the present invention, a transparent needleless injector drug capsule suitable for pre-filling with a liquid drug comprises a first inner layer of drug compatible transparent plastics defining a chamber for receiving a liquid drug and a second outer layer of transparent plastics forming a supporting sleeve around the first layer of plastics, wherein each of the first and second layers of transparent plastics is resistant to discolouration when irradiated by high energy radiation.

The present invention provides a low cost, transparent, needleless injector drug capsule which uses a plastics construction which is capable of being sterilised by high energy irradiation and thereby overcome the production problems associated with conventional glass and plastics drug capsules. Gamma irradiation causes glass and many plastics to assume a brown colour, which impairs visual inspection of the contents after filling. The selection of plastics materials which combine to provide the necessary drug compatibility and strength, and which are also suitable for gamma or other high energy irradiation, provides a cost effective solution to the problems of manufacturing conventional capsules.

As discussed above, an alternative material to glass suggested in the prior art is a transparent plastics, but there are very few suitable for long-term contact with most drugs. Those that are potentially chemically compatible have other drawbacks, such as poor resistance to irradiation, intense colouration as a result of irradiation, high water absorption, high gas or vapour transmission, or very low tensile strength. Generally, the plastics most suitable for drug contact are also very brittle, and whilst in theory a single layer, very thick walled capsule could be moulded to provide sufficient strength, this would result in severe post-moulding shrinkage and stress-induced micro-cracks. However, it has been found that it is possible to employ the optimum properties of one type of plastics, and add those of at least one other by moulding or assembling a multi-layered drug capsule, so that the complete capsule provides all of the necessary attributes, including resistance to discolouration and other detrimental changes in properties when irradiated. Sterilisation of drug capsules by gamma irradiation is significantly cheaper than methods currently used to sterilise glass or other plastics drug capsules.

Preferably, the first and second layers of plastics are injection moulded, with the first layer being bonded to the second layer at the interface. One reason for bonding the two materials is to prevent the formation of very small air gaps between layers, which would otherwise produce optical interference patterns (Newton's rings) and adversely affect visual or automatic inspection. Even a small gap would adversely affect the barrier properties of the combination. Accordingly, it is preferred that the second layer of plastics has a higher melting point than the first layer of plastics. The materials should have similar coefficients of expansion so that the bond layer doesn't become over stressed during cooling and any subsequent temperature fluctuations.

Preferably, the first layer of plastics is a metallocene catalysed polymer, most preferably a cyclic olefinic copolymer (COC) or a cyclic olefinic polymer (COP). This class of materials exhibits a number of useful properties making it suitable for long term drug contact, including extremely low water absorption, excellent water vapour barrier properties, high transparency, and low birefringence. The material can be sterilised by gamma irradiation without clouding or weakening. However, alone it is too brittle for use as a drug capsule in a needleless injector. In the present invention, the solution is to provide a tough impact resistant sleeve to lend support.

Preferably, the second layer of plastics is a polymer selected from a group consisting of polyesters, copolyesters, polyethylene naphthalate, polyamides, polycarbonates, and polyurethanes. These materials can provide a tough impact resistant transparent plastics support sleeve for the first layer of plastics and which may themselves be sterilised by gamma irradiation without clouding or weakening.

Preferably, the drug capsule further comprises a PTFE piston within the chamber for discharging the drug.

Preferably, the first layer of plastics is extended to form an integral filling adapter.

More preferably, the filling adapter includes a frangible tamper evident connection.

According to a second aspect of the present invention, a needleless injector comprises a drug capsule according to the first aspect of the present invention.

In one preferred example, the second or outermost layer of plastics is an integral part of the body of the needleless injector.

According to a third aspect of the present invention, a method of manufacturing a transparent drug capsule for a needleless injector comprises the steps of:

forming a multilayer capsule having a first inner layer of drug compatible transparent plastics and a second outer support layer of transparent plastics, each of the first and second layers of transparent plastics being selected so that they are resistant to discolouration when irradiated; and,

sterilising the multilayer capsule by high energy irradiation.

Preferably, the first layer of plastics is injection moulded and subsequently the second layer of plastics is moulded onto the first layer so that the two layers are bonded at the interface between them.

Preferably, the second layer of plastics has a higher melting point than the first layer of plastics. The materials should have similar coefficients of expansion so that the bond layer doesn't become over stressed during cooling and any subsequent temperature fluctuations.

Preferably, the drug capsule is preassembled with a PTFE piston located within the capsule, and the entire assembly is sterilised in a vacuum by high energy irradiation. PTFE is usually considered a radiation degradable polymer but it has been found that when irradiated in a vacuum, for example in a vacuum pack, sufficient strength is retained. Furthermore, it is preferred that the PTFE piston is pretreated by gamma or other high energy radiation at an elevated temperature. This treatment causes crosslinking, with a consequent increase in strength and resistance to further irradiation.

Preferably, the method further comprises the step of filling the sterilised drug capsule with a liquid drug in an automated process and subsequently sealing the capsule in a manner suitable for transport and long term storage.

According to a fourth aspect of the present invention, a drug capsule for a needleless injector comprises a body having a main chamber for receiving a liquid drug and for retaining a free piston in a sealing fit for subsequent use in the discharge of a drug, the body also having an extension chamber having an opening for receiving the free piston in a loose fit.

In this aspect of the present invention, a multichamber drug capsule is provided which allows a free piston, typically a PTFE piston, to be preassembled within it in a loose fit. Since the piston is initially assembled in a loose fit, and therefore not under mechanical stress, it would not be degraded by normal levels of gamma radiation. In any case, the clearance provided by the initial loose fitting of the piston allows penetration by a sterilization fluid. Accordingly, the drug capsule is capable of being assembled in clean conditions and subsequently sterilized by any known method. Once sterilized, the free piston is pushed into position prior to filling so that it is located and retained in a sealing fit within the main chamber for subsequent use in the discharge of a drug.

Preferably, the drug capsule further comprises a stop to retain the free piston within the extension chamber. More preferably, the stop comprises a number of integral stakes formed by thermal or ultrasonic displacement of material at the opening of the extension chamber. Alternatively, the stop could be a separate fitting which is connected to the opening of the extension chamber.

Preferably, the extension chamber comprises a tapered section. The extension chamber may be tapered over its entire length or alternatively comprise a parallel section and a tapered section, the tapered section being provided at a transition between the main chamber and the extension chamber.

Preferably, the drug capsule is a transparent plastics drug capsule in accordance with the first aspect of the present invention.

According to a fifth aspect of the present invention, the combination of a drug capsule in accordance with the fourth aspect of the present invention and a free piston.

Preferably, the free piston is manufactured from PTFE.

According to a sixth aspect of the present invention, a method of manufacturing a drug capsule comprises the steps of:

forming a drug capsule in accordance with the fourth aspect of the present invention;

assembling a free piston in the extension chamber in a loose fit;

sterilising the drug capsule assembly; and,

locating the free piston within the main chamber in a sealing fit.

Preferably, the drug capsule is then filled. The piston may either be pushed to the discharge end and the capsule then filled with injectate thereby returning the piston to the other end of the main chamber under the pressure of the injectate. Alternatively, the injectate may be introduced by first evacuating the volume of the main chamber and then filling the main chamber with the injectate.

Sterilisation may be carried out using a fluid. Preferably, the fluid is steam or ethylene oxide. Alternatively, the drug capsule may be sterilized by exposure to high energy radiation.

The drug capsule may be formed of glass. Preferably, however, the drug capsule is a plastics drug capsule.

According to a seventh aspect of the present invention, a needleless injector comprises a drug capsule in accordance with the fourth aspect of the present invention.

### BRIEF DESCRIPTION OF THE DRAWINGS

Examples of the present invention will now be described in detail with reference to the accompanying drawings, in which:

FIG. 1 shows a first example of a plastics drug capsule in accordance with the present invention;

FIGS. 2 and 3 show the filling of the drug capsule of FIG. 1;

FIG. 4 shows the drug capsule of FIG. 1 assembled to the body of a needleless injector;

FIG. 5 shows an example of a needleless injector incorporating a second example of a plastics drug capsule in accordance with the present invention;

FIG. 6 shows a third example of a drug capsule in accordance with the present invention; and,

FIGS. 7 and 8 show the filling of the drug capsule of FIG. 6.

### DETAILED DESCRIPTION

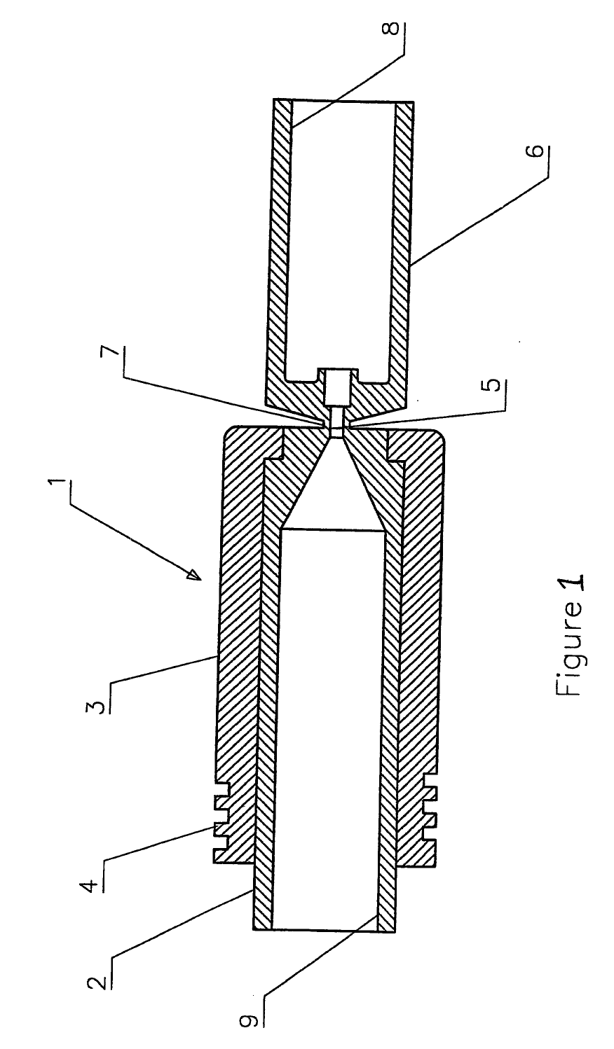

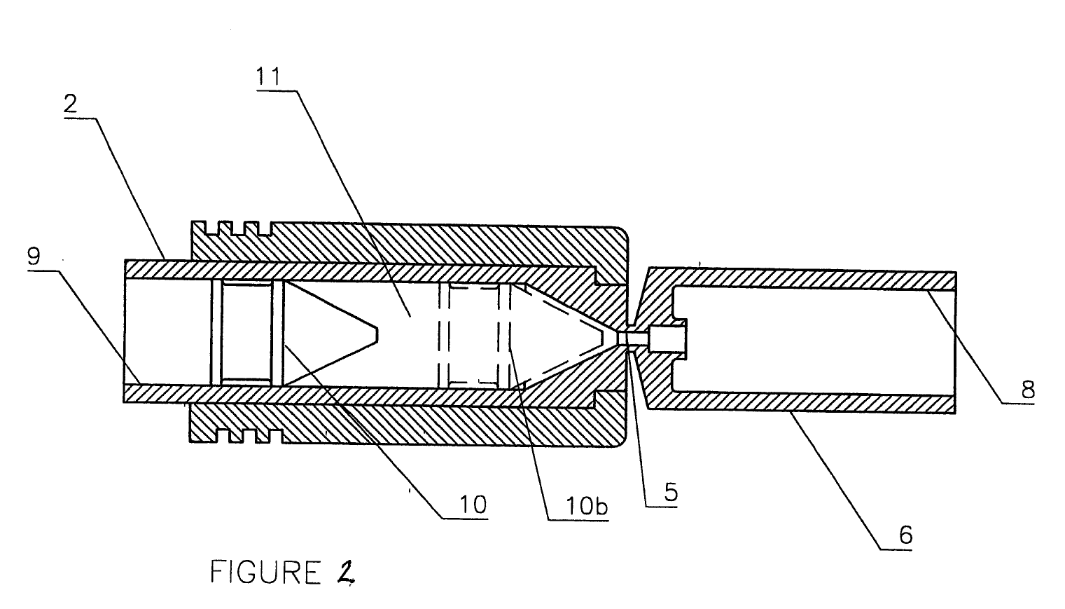

Referring to FIG. 1 which is a centre-line section through a cylindrical capsule, the capsule 1 comprises an inner liner 2 and a sleeve 3. A screw thread 4 for attaching the capsule to an actuator is provided, although a snap fit, bayonet or other well-known connection may be used as an alternative. The liner 2 has an orifice 5 at one end through which the injectate is dispensed, and is in frangible and hydraulic connection at 7 with the filling connector 6. The materials of the liner 2 and connector 6 are preferably the same, and suitable for contact with the drug. The bore 9 of the liner 2 is substantially parallel, and has a surface finish to be compatible with a material of a piston (see FIG. 2).

In FIG. 2, the capsule 1 is shown with a piston 10 assembled therein. The piston 10 seals and slides in the bore 9 of the liner 2 and is of a quality to prevent microbial contamination of the drug. Piston 10 is shown in position suitable for vacuum filling, whereby the volume 11 is evacuated via orifice 5, and then filled with liquid drug through the orifice 5. The friction of the piston 5 within the bore 9 is sufficient to prevent its movement during evacuation. An alternative to vacuum filling is to position the piston as at 10b, in which case the small void is first evacuated and the drug then introduced through orifice 5 at a pressure sufficient to force the piston 10 along the bore 9 to the required position. The configuration of the connector 6 may be adapted to suit the filling machine, and a suitable filling method is described in our co-pending application PCT/GB96/03017.

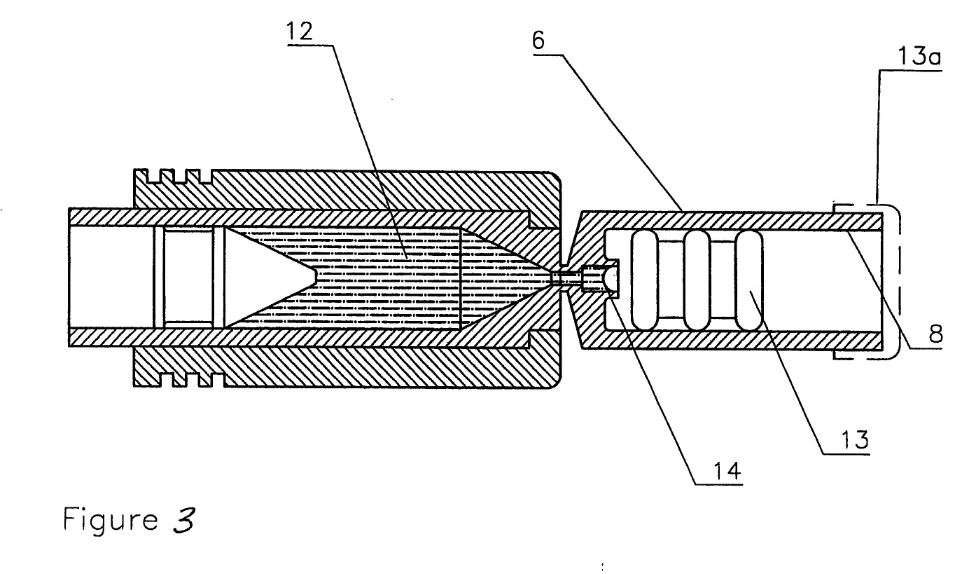

FIG. 3 shows the capsule described above filled with a liquid drug 12 and sealed. The drug 12 is filled as described, with a small excess in chamber 14 to permit thermally induced volume fluctuations. An elastomeric seal 13 is placed in the bore 8 of connector 6, or (not shown) a small sealing plug may be inserted in the chamber 14. Alternative methods of sealing are a cap 13a, or by thermally softening the walls of the connector 6 and crimping to provide an hermetic seal. There are still other sealing methods such as ultrasonically welding and radio frequency bonding of a foil membrane which may be used, the object in each case to effect a seal against microbial contamination.

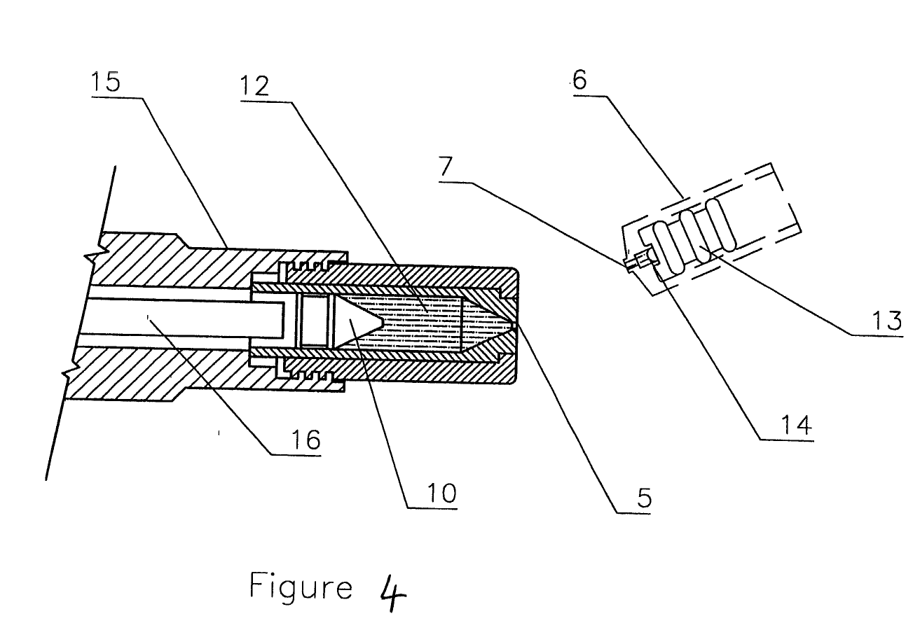

FIG. 4 shows the capsule 1 assembled to an injector actuator 15, and connector 6 together with the seal 13 snapped off at the frangible connection 7, exposing the orifice 5 of the capsule 1. The injector is thus prepared for giving an injection by placing the orifice 5 onto the patient's skin, and operating the actuator to release the stored energy therein, which causes the push-rod 16 to act on piston 10 and dispense the injectate 12 through the orifice 5.

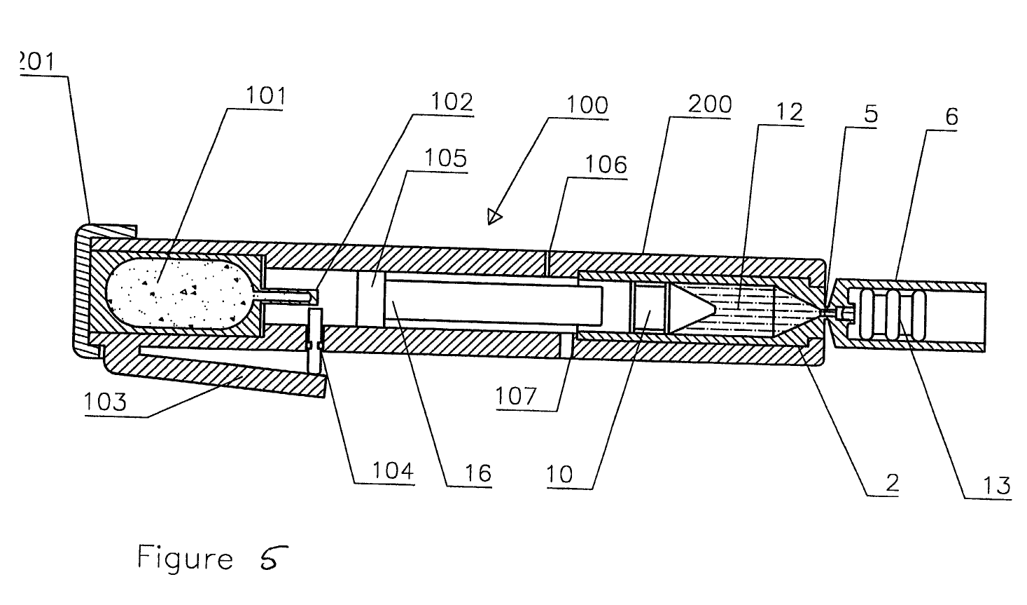

A complete injector 100 is shown in FIG. 5. In this example, the liner 2 is moulded integrally with the body 200, and is of a similar form to that described above. The energy source is a compressed carbon dioxide cartridge 101 (although other liquified gases may be used) having a frangible tube 102. A cap 201 welded or otherwise fixed to body 200 retains the cartridge 101 in situ. This injector is operated by removing the filling connector 6 together with seal 13, and pressing the orifice 5 against the skin. Acting on the lever 103 to push rod 104 against the frangible tube 102 sufficiently to snap the tube 102 causes the release of carbon dioxide gas from the cartridge. The piston 105 is attached to a push-rod 16, and the compressed gas acts on the piston 105 to drive it forward to first strike the piston 10 to create a rapid pressure rise, and then to continue to push the piston 10 to discharge the injectate. A hole 107 prevents excessive back-pressure which would reduce the force of the piston 105, and a hole 106 is positioned so that the piston 105 just passes it at the end of the injection to exhaust residual compressed carbon dioxide. Instead of compressed gas, a compressed coil spring or gas spring may be used, and the simple energy release trigger may be replaced by a release mechanism that operates in response to pressure of the orifice on the skin. The latter feature is desirable since it reduces the skill required to achieve a reproducible contact force on the skin, which has a direct effect on injection quality.

The material for the inner liner of the capsule must be compatible with the drug and the material for the outer sleeve must be tough and impact resistant. Both materials should be susceptible to sterilisation. Furthermore, the properties of both materials should enable a bond to form at the interface.

The preferred material for the inner liner is a cyclic olefin copolymer (COC) known as Topaz (RTM) and manufactured by Ticona (Germany). This material is a metallocene-catalysed polymer of ethylene (CH₂═CH₂) and 2-norbornene. In Topaz (RTM), the bulky norbornene group stiffens the chain resulting in a totally amorphous structure where no melting point is observed. A particularly preferred grade of Topaz (RTM) is Topas 6015 (RTM) which is a clear general propose grade with a heat deflection temperature HDT/B of 150° C. These metallocene-catalysed cyclolefin copolymers exhibit high optical qualities and have low levels of extractable materials. They have a water absorption (24 hour immersion in water at 23° C.) according to ISO 62 of less than 0.01%, a tensile strength according to ISO 527 parts 1 and 2 of 66 N/mm², an elongation at break of 4% and a notched impact strength (Charpy) according to ISO 179-1eA of 2.0 kJ/m². This material can be stabilized by various sterilisation methods known in the art such as sterilisation using steam, ethylene oxide gas and gamma irradiation. Using gamma irradiation at 30 kGy, no change in mechanical properties is observed and only minor changes in the optical properties are observed. The change in yellow index measured according to DIN 6187 (ΔYI) is 1-2 and the change of the haze measured according to ASTM 1003 (ΔHaze) is less than 0.4. Topaz (RTM) can be processed on conventional injection moulding machines.

Typical plastics which can be moulded onto the inner layer are copolyesters such as Eastar GN007 manufactured by Eastman (USA), polyethylene napthalate, polyurethanes, nylon 12 and polycarbonate. A particularly preferred material for the outer layer is a specific grade of polycarbonate, Makrolon Rx-1805 (RTM) with colour additive 45/311 manufactured by Bayer (Germany). This material is a transparent high viscosity polycarbonate based on Bisphenol A with a specialised additive system. This material has high chemical resistance and resistance to gamma irradiation. It has a tensile modulus according to ISO 527 of 2400 MPa and a IZOD notched impact strength at 23° C. according to ISO 180-4A of 95 KJ/m² Makrolon Rx-1805 (RTM) can be sterilised by the usual methods, for example with steam, ethylene oxide gas or gamma irradiation. It has high colour stability after gamma irradiation. This material can be processed on all modern injection moulding machines.

In a preferred embodiment of the present invention, a drug capsule is manufactured using Topaz 6015 (RTM) as the inner layer and Makrolon Rx-1805 (RTM) as the outer layer. Any conventional injection moulding machine can be used. However, preferred processing parameters are as follows. For Topaz 6015 (RTM), injection moulding should be carried out at a tool temperature of 110-150° C., an injection speed of 5-40 cm³/s, a shot volume of 27-30 cm³, an injection time of 0.80 s, a holding pressure of 200-600 bar, a holding time of 2-3.5 s and a back pressure of 60 bar. For Makrolon Rx-1805 (RTM) the preferred processing parameters are a barrel temperature of 240-300° C., a mould temperature of 80-120° C., an injection pressure of 500-1000 bar, an injection time of 0.5-1.0 s, a back pressure of 20-100 bar, a holding pressure of 150-450 bar and a holding time of 1.5-3.0 s.

Normally the liner would be moulded first, allowed to cool slightly, and the outer layer then moulded around it. The liner would typically have a thickness of 1-2 mm, preferably 1.5 mm, and the outer layer a thickness of 2-4 mm, preferably 3 mm. Preferably, the temperatures should be selected so that the second moulding slightly melts and bonds to the first and the inner liner formed thereafter. Of course, the design of the capsule or injector may dictate that the outer part is moulded first. Again, it is important that the coefficients of expansion of the two materials are in the correct range, so that there are no large stresses produced as a result of differential thermal expansion. Additional layers or features may be moulded onto the capsule, such as plastics that change colour when irradiated to provide a visual indication that the device has been sterilised.

The inner layer may also be formed from a liquid crystal polymer or from a poly-para-xylylene (parylene). These materials are suitable for long term drug contact and are opaque or translucent when moulded in thin layers.

Preferred liquid crystal polymers are selected from the aromatic copolyesters exemplified by commercial products such as Vectra (RTM) (Hoechst-Celanese), Xydan (RTM) (Amoco Performance Products), HX type liquid crystal polymers (DuPont), Eikonol (RTM) and Sumikasuper (RTM) (Sumitomo Chemical), Rodrun (RTM) (Unitika) and Granlar (RTM). A thin layer of liquid crystal polymer may be obtained by co-extending the liquid crystal polymer with the plastics material forming the outer layer of the drug capsule, such as polycarbonate, for example. This results in a tubular form which is thermo-formed into a desired shape.

Parylene coatings are formed from an active monomer gas which is capable of polymerising on the surface of the pre-moulded outer sleeve. It may be formed in layers typically from a few molecules to 75 microns thick.

A preferred material for the piston is polytetrafluoroethylene (PTFE), which has low stiction (coefficients of dynamic and static friction are similar) and excellent chemical resistance. It is easily deformed so that a piston made from the material may be inserted to provide an interference fit in the capsule bore, yet it has a high modulus of elasticity at the high strain rates induced during the first part of the injection. The latter property ensures a high coefficient of restitution and maximum transfer of energy from the actuator into the drug. It should be noted that a number of prior art injectors specify the use of ‘O’ ring seals for the piston: these have a number of drawbacks, one of which is that they are known to “weld” to the bore of the capsule when in long term contact without a lubricant or release agent at the interface. The traditional remedy is to coat the capsule body with a silicone emulsion and bake it to form a very thin layer of lubricant. Alternatively, the ‘O’ ring may be siliconised. The baking process is not suitable for plastics and therefore the only option in this case is to use a treated ‘O’ ring. Highly transparent plastics are amorphous and the types most suitable for storing drugs are very sensitive to contact with oils and craze when contaminated. Another problem with ‘O’ rings is that the minute movement of the rings in their grooves at the start of the injection increases the rise time of the pressure pulse in the drug. A potential drawback of using PTFE is that it loses most of its strength when irradiated to a typical doses of 25 kGys (2.5 Mrads) used in a gamma sterilisation process. Also, it has been found that when PTFE is subjected to irradiation whilst under mechanical stress it deforms to relieve that stress. Thereafter, a piston that has been irradiated within the bore could be loose or have insufficient sealing within the bore. Accordingly, it is preferred that the PTFE piston is pretreated by gamma or other high energy radiation at high temperature. This treatment has been found to cause crosslinking, with a consequent increase in strength and resistance to further irradiation.

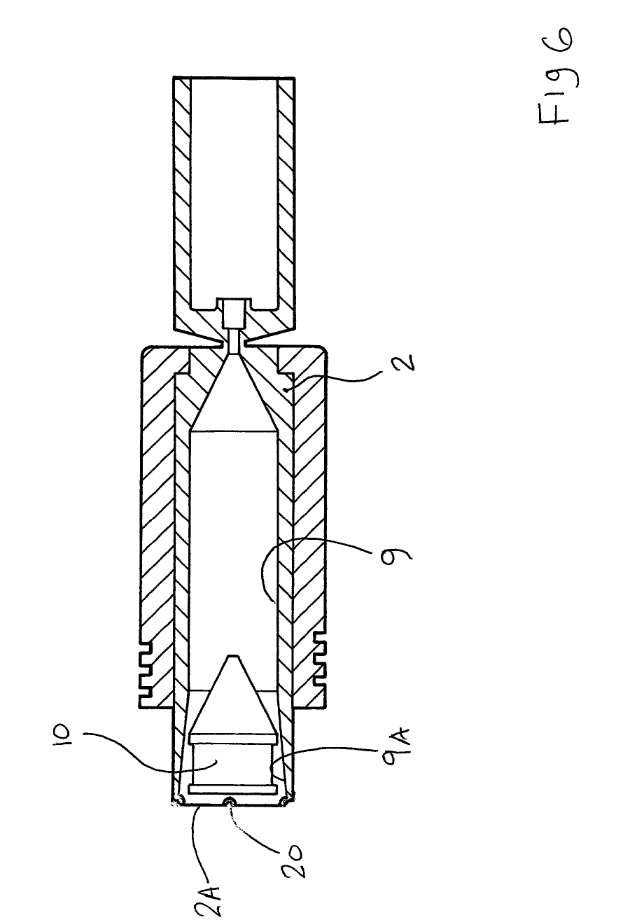

FIG. 6 shows another example of a drug capsule. The capsule 2 has a bore 9, enlarged at the open end at 9A. Bore 9A is shown as a taper, for ease of manufacture, but it may be a parallel bore connected to bore 9 by a short tapered section. The piston 10 is a loose fit within the bore 9A and is retained therein by staking the rim 2A of capsule 2. Other suitable retaining means may be used instead. The staking 20 may be formed by thermal or ultrasonic displacement of the rim 2A.

The preferred material for the piston 10 is PTFE, and because the piston 10 is a loose fit within the bore 9A, and therefore not under mechanical stress, it would not be degraded by normal levels of gamma radiation, ie up to 40 kGys. In any case, the clearance between the piston 10 and bore 9A allows penetration of steam for autoclaving or gasing by ethylene oxide, both common alternatives for sterilising devices for parenteral delivery of drugs. Thus, the piston and capsule may be assembled in clean conditions and subsequently sterilised by any known method.

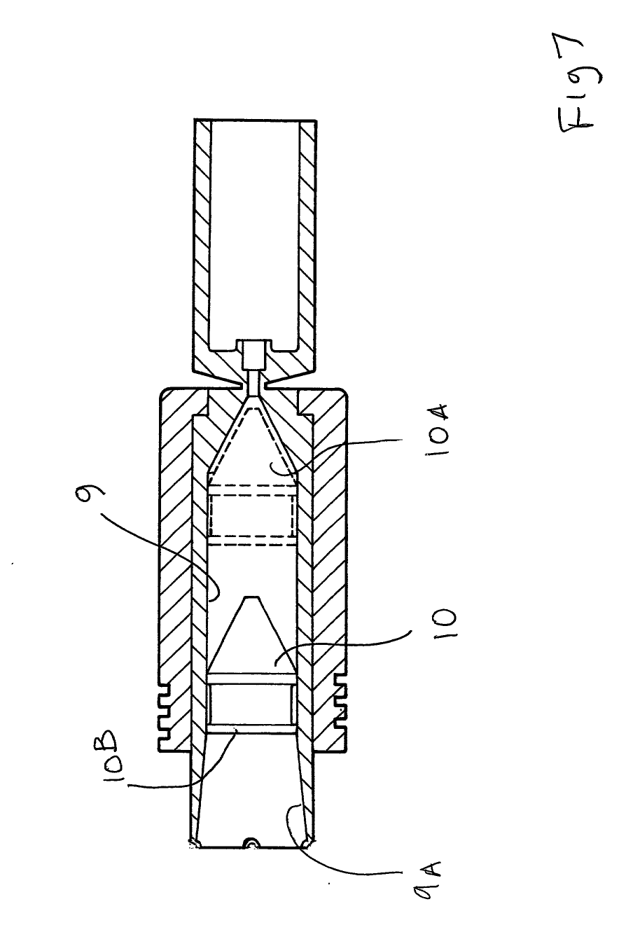

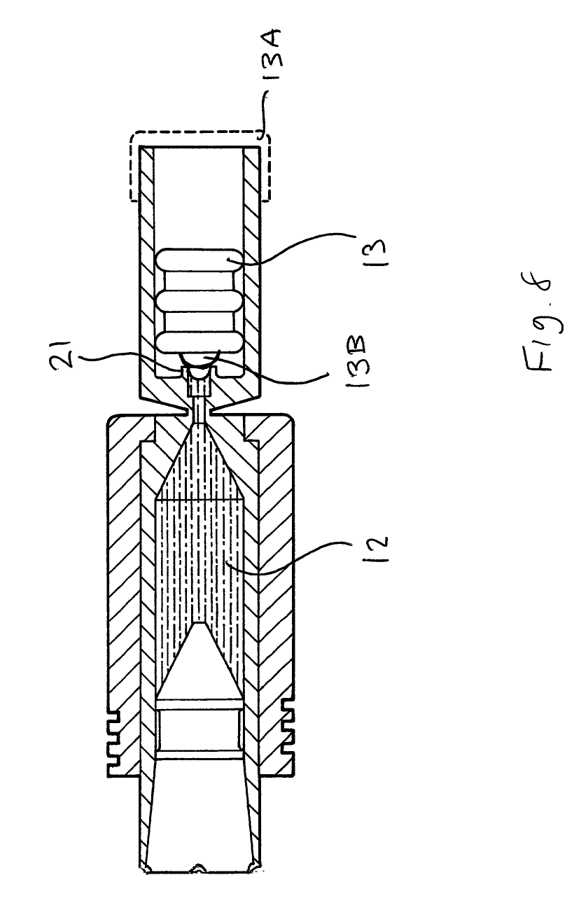

After sterilisation, the capsule is filled. As shown in FIG. 7, the piston 10 is pushed from the enlarged bore 9A into the substantially parallel bore 9, so that the ribs 10b make sealing contact with the bore 9. Alternatively, the piston may be pushed to the discharge end of the capsule at 10A. The capsule is then filled with injectate 12, as shown in FIG. 8, and sealed by a plug 13 or cap 13A. The plug 13 may have a projection 13B which seals on the filling orifice 21.

In the present application, the term ‘plastics’ is used in the generic sense as for certain organic substances, mostly synthetic or semi-synthetic (casein and cellulose derivatives) condensation or polymerisation products, and also certain natural substances (shellac, bitumen, but excluding natural rubber), which under heat and pressure become plastic, and can then be shaped or cast in moulds, or extruded.

We claim:

1. A transparent needleless injector drug capsule suitable for prefilling with a liquid drug, comprising a first inner layer of drug compatible transparent plastics defining a chamber for receiving a liquid drug and a second outer layer of transparent plastics forming a supporting sleeve around the first layer of plastics, wherein each of the first and second layers of transparent plastics is resistant to discolouration when irradiated by high energy radiation.

2. A needleless injector according to claim 1, in which the first and second layers of plastics are injection moulded, with the first layer being bonded to the second layer at the interface.

3. A needleless injector drug capsule according to claim 1, in which the second layer of plastics has a higher melting point than the first layer of plastics.

4. A needleless injector drug capsule according to claim 1, in which the first layer of plastics is a metallocene catylised polymer.

5. A needleless injector drug capsule according to claim 1, in which the first layer of plastics is a cyclic olefinic copolymer.

6. A needless injector drug capsule according to claim 1, in which the second layer of plastics is a polymer selected from a group consisting of polyesters, copolyesters, polyethylene naphthalate, polyamides, and polyurethanes.

7. A needleless injector drug capsule according to claim 1, further comprising a polytetrafluorethylene piston within the chamber for discharging the drug.

8. A needleless injector drug capsule according to claim 1, in which the first layer of plastics is extended to form an integral filling adapter.

9. A needleless injector drug capsule according to claim 8, in which the filling adaptor includes a frangible tamper evident connection.

10. A needleless injector drug capsule according to claim 1, comprising a body having a main chamber for receiving a liquid drug and for retaining a free piston in a sealing fit for subsequent use in the discharge of a drug, the body also having an extension chamber having an opening for receiving the free piston in a loose fit.

11. A needleless injector drug capsule according to claim 10, further comprising a stop to retain the free piston within the extension chamber.

12. A needleless injector drug capsule according to claim 11, in which the stop comprises a number of integral stakes formed by thermal or ultrasonic displacement of material at the opening of the extension chamber.

13. A needleless injector drug capsule according to claim 11, in which the stop comprises a separate fitting which is connected to the opening of the extension chamber.

14. A needleless injector drug capsule according to claim 10, in which the extension chamber comprises a tapered section.

15. A needleless injector drug capsule according to claim 14, in which the extension chamber is tapered over its entire length.

16. A needleless injector drug capsule according to claim 14, in which the extension comprises a parallel section and a tapered section, the tapered section being provided at a transition between the main chamber and the extension chamber.

17. A needleless injector comprising a drug capsule according to claim 1.

18. A needleless injector according to claim 17, in which the second or outermost layer of plastics is an integral part of the body of the needleless injector.

19. A method of manufacturing a transparent drug capsule for a needleless injector, comprising the steps of:
forming a multi-layer capsule having a first inner layer of drug compatible transparent plastics and a second outer support layer of transparent plastics, each of the first and second layers of transparent plastics being selected so that they are resistant to discolouration when irradiated; and,
sterilising the multi-layer capsule by high energy irradiation.

20. A method according to claim 19, in which the first and second layers of plastics are injection moulded so that the two layers are bonded at the interface between them.

21. A method according to claim 19, in which the second layer of plastics has a higher melting point than the first layer of plastics.

22. A method according to claim 19, in which the drug capsule is preassembled with a polytetrafluoroethylene piston located within the capsule.

23. A method according to claim 22, in which the polytetrafluoroethylene piston is pretreated by exposure to high energy radiation at an elevated temperature.

24. A method according to claim 19, further comprising the step of filling the sterilized drug capsule with a liquid drug in an automated filling process and subsequently sealing the capsule in a manner suitable for transport and long term storage.

25. A drug capsule for a needleless injector comprising a body having a main chamber for receiving a liquid drug and for retaining a free piston in a sealing fit for subsequent use in the discharge of a drug, the body also having an extension chamber having an opening for receiving the free piston in a loose fit.

26. A drug capsule according to claim 25, further comprising a stop to retain the free piston within the extension chamber.

27. A drug capsule according to claim 26, in which the stop comprises a number of integral stakes formed by thermal or ultrasonic displacement of material at the opening of the extension chamber.

28. A drug capsule according to claim 26, in which the stop comprises a separate fitting which is connected to the opening of the extension chamber.

29. A drug capsule according to claim 25, in which the extension chamber comprises a tapered section.

30. A drug capsule according to claim 29, in which the extension chamber is tapered over its entire length.

31. A drug capsule according to claim 29, in which the extension comprises a parallel section and a tapered section, the tapered section being provided at a transition between the main chamber and the extension chamber.

32. A transparent needleless injector drug capsule according to claim 25, suitable for prefilling with a liquid drug, comprising a first inner layer of drug compatible transparent plastics defining a chamber for receiving a liquid drug and a second outer layer of transparent plastics forming a supporting sleeve around the first layer of plastics, wherein each of the first and second layers of transparent plastics is resistant to discolouration when irradiated by high energy radiation.

33. A needleless injector according to claim 32, in which the first and second layers of plastics are injection moulded, with the first layer being bonded to the second layer at the interface.

34. A needleless injector drug capsule according to claim 32, in which the second layer of plastics has a higher melting point than the first layer of plastics.

35. A needleless injector drug capsule according to claim 32, in which the first layer of plastics is a metallocene catylised polymer.

36. A needleless injector drug capsule according to claim 32, in which the first layer of plastics is a cyclic olefinic copolymer.

37. A needless injector drug capsule according to claim 32, in which the second layer of plastics is a polymer selected from a group consisting of polyesters, copolyesters, polyethylene naphthalate, polyamides, and polyurethanes.

38. A needleless injector drug capsule according to claim 32, further comprising a polytetrafluorethylene piston within the chamber for discharging the drug.

39. A needleless injector drug capsule according to claim 32, in which the first layer of plastics is extended to form an integral filling adapter.

40. A needleless injector drug capsule according to claim 32, in which the filling adaptor includes a frangible tamper evident connection.

41. A needleless injector comprising a drug capsule according to claim 25.

42. In combination, a drug capsule according to claim 25 and a free piston.

43. The combination according to claim 42, wherein the free piston is manufactured from PTFE.

44. A method of manufacturing a drug capsule comprising the steps of:
forming a drug capsule for a needleless injector comprising a body having a main chamber for receiving a liquid drug and for retaining a free piston in a sealing fit for subsequent use in the discharge of a drug, the body also having an extension chamber having an opening for receiving the free piston;
assembling a free piston in the extension chamber in a loose fit;
sterilising the drug capsule assembly; and,
locating the free piston within the main chamber in a sealing fit.

45. A method according to claim 44 further comprising filling the drug capsule.

46. A method according to claim 44, in which the piston is pushed to the discharge end and the capsule then filled with injectate thereby returning the piston to the other end of the main chamber under the pressure of the injectate.

47. A method according to claim 44, in which the injectate is introduced by first evacuating the volume of the main chamber and then filling the main chamber with the injectate.

48. A method according to claim 44, in which the step of sterilising comprises fluid sterilisation.

49. A method according to claim 48, in which the fluid for sterilisation is steam or ethylene oxide.

50. A method according to claim 44, in which the step of sterilising comprises exposure to high energy radiation.

51. A method according to claim 44, in which the capsule is formed of glass.

52. A method according to claim 44, in which the capsule is formed of plastics.
